(19) United States
(12) Patent Application Publication
(10) Pub. No.: US 2014/0212940 A1
(43) Pub. Date: Jul. 31, 2014

(54) MEMBRANES

(71) Applicants: Weishen Yang, Liaoning (CN); Xinlei Liu, Liaoning (CN); Yanshuo Li, Liaoning (CN)

(72) Inventors: Weishen Yang, Liaoning (CN); Xinlei Liu, Liaoning (CN); Yanshuo Li, Liaoning (CN)

(73) Assignees: BP P.L.C., London (GB); Dalian Institute of Chemical Physics, Chinese Academy of Sciences, Liaoning (CN)

(21) Appl. No.: 14/119,723

(22) PCT Filed: May 25, 2011
(86) PCT No.: PCT/CN2011/000896

§ 371 Date: Feb. 28, 2014

(51) Int. Cl. B01D 71/64; C07C 31/12

(52) U.S. Cl. CPC B01D 71/64; C07C 31/12
USPC 435/157; 210/640; 210/500.25; 568/913; 427/244

(57) ABSTRACT

Mixed matrix pervaporation membranes are described which include i) a matrix phase comprising a polymeric material, and ii) a zeolitic imidazolate framework (ZIF) dispersed in the matrix phase. In membranes described, the thickness of the membrane is greater than 0.5 μm. The membranes may in examples be used in a process for separating an organic compound from an aqueous liquid mixture. An example process includes contacting the liquid mixture on one side of a mixed matrix pervaporation membrane to cause the organic compound to permeate the mixed matrix membrane, and removing from the other side of the membrane a permeate composition comprising a portion of the organic compound which permeated the membrane. Example membranes described have relatively good selectivity for separation of the organic compound from the liquid mixture.

This invention relates to membranes. Aspects of the invention relate to organic-inorganic membranes. In some examples of the invention, the membranes are suitable for use as pervaporation membranes. Aspects of the invention relate to membranes including metal-organic frameworks (MOFs), for example to pervaporation membranes including MOFs, to methods for their manufacture and their applications. Examples of aspects of the invention relate to membranes for use in pervaporation for recovering organic compounds from a solution, for example an aqueous solution. Examples of aspects of the invention find particular, but not exclusive, application in relation to the recovery of alcohol, for example butanol, from solution, for example aqueous solution. Examples of aspects of the invention find particular, but not exclusive, application in relation to the separation of alcohols, for example from fermentation broth in a fermentation process for the production of alcohol from biological sources. In examples, membranes of aspects of the present invention may find application in relation to the separation of butanol from fermentation broth.

Alcohols, for example ethanol and butanol, are widely used as biofuels, solvents, and/or as precursors for chemical synthesis. With currently known fermentation techniques for the production of alcohols, the final concentration of butanols in fermentation broths is generally low, for example about 100 g/L of ethanol, and only about 20 g/L of iso-butanol in some examples. Distillation is a traditional recovery option for butanol, but is very energy intensive, in particular in view of the low yield of butanol.

A reason for the low yield of alcohols from fermentation processes is that the produced alcohols may be toxic to fermentation organisms above a critical concentration and the fermentation process may substantially stop above this concentration. Simultaneous separation of product from the fermentation reactor can make the fermentation proceed in a substantially continuous manner, reducing downtime and improving the productivity of the reactor.

From an energy requirement perspective, pervaporation is presently considered to be one of the more attractive options for effecting separation of product from the fermentation reactor. Pervaporation involves the separation of mixtures of liquids by partial vaporization through a membrane. The separation of the components is based on a difference in transport rate of individual components through the membrane. The efficiency of the separation, for example including rate of separation (flux) and selectivity (separation factor) of the separation to particular components, depends on the chemical and physical properties of the membrane.

Materials of pervaporative membranes for recovering alcohols currently studied include polydimethylsiloxane (PDMS) and poly(1-trimethylsilyl-1-propyne) (PTMSP). PDMS is currently considered to be a benchmark membrane material due to its performance in the recovery of alcohol. The reported butanol-water separation factor for a PDMS membrane ranges from 2.4 to 44.0, with a flux of several tens of gm⁻²h⁻¹.

There is a need for an improved membrane technology for separation of butanol from fermentation broth. There is also a need for improved membranes for use in the separation of other alcohols and/or other organic materials from solution. Improved membranes would preferably exhibit improved flux and/or separation factor compared with known membrane technologies.

It has been reported that the addition of zeolite into a PDMS membrane as a filler can increase the selectivity of the membrane by forming a composite membrane. Composite membranes using zeolites are described for example in Journal of Membrane Science 192 (2001) 231-242. Unfortunately, there are some drawbacks to the use of zeolites in composite membranes. For example, the number of zeolite topologies and compositions has been found to be limited; the preparation of ultrafine defect-free zeolite crystals is costly, difficult and time-consuming; the dispersion of zeolites and zeolite-polymer contact are sometimes not good. Consequently, alternative filler materials having improved properties compared with the use of zeolites are desirable.

Metal-organic frameworks (MOFs) form a family of molecular sieves consisting of metal ions or clusters interconnected by a variety of organic linkers. MOFs possess interesting properties, such as highly diversified structures, high surface areas, large range in pore sizes and specific adsorption affinities. These make MOFs attractive candidates for using as fillers in the construction of mixed matrix membranes (MMMs). In the past few years, attempts at fabricating MOFs-containing membranes have been carried out. MOFs-containing separate membranes (including supported pure MOFs membranes) have been reported for use in gas separation, for example, as described in Journal of Membrane Science. 361 (2010) 28-37, U.S. Pat. No. 7,637,983, and Angewandte Chemim International Edition. 49, (2010) 548-551, Journal of the American Chemical Society. 131 (2009) 16000-16001.

The single-crystal adsorbent [Cu^{II}₂(bza)₄(pyz)]ₙ mixed silicone rubber membrane reported by Takamizawa et al. (Chemistry an Asian Journal. 2 (2007) 837-848) has found application in pervaporation. This membrane (300 μm thick) was reported to exhibit an ethanol/water separation factor of 6.2 and total flux of 0.047 kgm⁻²h⁻¹ towards a 5 wt. % alcohol aqueous solution at 25° C. Another MOFs-containing (mixed matrix) membrane used for liquid separation was applied in nanofiltration reported by Vankelecom et al. (Journal of Membrane Science. 344 (2009) 190-198).

It would be desirable to provide a membrane which has a relatively high flux and good selectivity for the separation of organic compounds, for example from an aqueous liquid mixture.

According to a first aspect of the invention there is provided a process for separating an organic compound from an aqueous liquid mixture, the process comprising:

a) contacting the liquid mixture on one side of a mixed matrix pervaporation membrane to cause the organic compound to permeate the mixed matrix membrane, wherein the mixed matrix pervaporation membrane comprises

i) a matrix phase comprising a polymeric material, and

ii) a zeolitic imidazolate framework (ZIF) material dispersed in the matrix phase

wherein the thickness of the mixed matrix pervaporation membrane is greater than 0.5 μm and
b) removing from the other side of the membrane a permeate composition comprising a portion of the organic compound which permeated the membrane.

Zeolitic imidazolate frameworks (ZIFs) are a subfamily of MOFs. ZIF structures are generally based on the structures of aluminosilicate zeolites. Aluminosilicate zeolites include tetrahedral Si or Al atoms with bridging O atoms. ZIFs include metal ions, for example transition metal ions, with imidazolate linkers. For example, ZIFs are constructed from tetrahedral metal ions, for example Zn and/or Co, bridged by imidazolate. A description of ZIFs and their use and preparation is described for example in US Patent Application No. 2010/0186588, International Patent Application No. WO2007/101241 and International Patent Application No. WO2008/140788.

A number of ZIFs are known to exhibit good thermal and chemical stability. Moreover, some ZIFs possess hydrophobic surfaces. Hence, it has been identified by the inventors that ZIFs and in particular hydrophobic ZIFs may be of use in pervaporative recovery of organic compositions from aqueous solutions. In examples of the invention, ZIFs may be provided in mixed silicone rubber membranes in applications and use in pervaporative recovery of organics from aqueous solutions.

International Patent Application No. WO2010/012660 describes the production of alcohol from a hydrocarbonaceous compound in the presence of a microbiological organism. The alcohol produced is absorbed from the produced solution by contacting the solution with a ZIF absorbent. The ZIF absorbent may for example be in the form of particles. Alcohol-comprising ZIF absorbent is subsequently isolated from the produced solution and treated to desorb the alcohol from the ZIF. International Patent Application No. WO2010/012660 describes the use of ZIFs in composite membranes having a membrane thickness of 0.5 μm

The inventors have identified that the thickness of the membrane is of particular importance. It has been identified that relatively thin membranes, for example having a thickness of less than 0.5 μm, are more likely to exhibit undesirable swelling in use, and may be more stable than relatively thicker membranes.

The thickness of the membrane may in some examples be substantially the same throughout the membrane. In other examples, there may be variation in the membrane thickness across the membrane. In such cases, where reference is made herein to a membrane thickness, preferably the majority of the membrane has that thickness. Where the membrane is used in the process for separating the organic compound, preferably where reference is made herein to a membrane thickness, preferably substantially all of the membrane contacting the liquid mixture has that thickness.

According to an aspect of the invention, the thickness of the membrane is greater than 0.5 μm, preferably greater than 0.7 μm, for example greater than 1.0 μm, for example greater than 2.0 μm.

Preferably the ZIF is dispersed in the polymer matrix phase. At least a part of the ZIF may be for example embedded in the polymer matrix phase. In examples of the invention the membrane may include ZIF particles with the polymer matrix phase at least partly filling interspaces between the ZIF particles. The polymer matrix phase may at least partly fill pores in the ZIF particles.

The membrane includes one or more ZIFs.

Preferably the membrane includes a hydrophobic ZIF.

Preferably the one or more ZIFs in the membrane includes one or more selected from the hydrophobic group comprising ZIF-1, ZIF-2, ZIF-3, ZIF-4, ZIF-5, ZIF-6, ZIF-7, ZIF-8, ZIF-9, ZIF-10, ZIF-11, ZIF-12, ZIF-14, ZIF-20, ZIF-21, ZIF-22, ZIF-23, ZIF-25, ZIF-60, ZIF-61, ZIF-62, ZIF-63, ZIF-64, ZIF-65, ZIF-66, ZIF-67, ZIF-68, ZIF-69, ZIF-70, ZIF-71, ZIF-72, ZIF-73, ZIF-74, ZIF-75, ZIF-76, ZIF-78, ZIF-90, ZIF-91, ZIF-92, ZIF-93, ZIF-96, ZIF-97 and ZIF-100.

For example, the ZIF(s) may include ZIF-4, ZIF-7, ZIF-8, ZIF-10, ZIF-22, ZIF-69, ZIF-78 and/or ZIF-90.

The membrane may include more than one ZIF, for example as a mixture.

The membrane may include at least 1 wt % ZIF. For example, the membrane may include at least 5 wt %, for example at least 10 wt %. For example, the membrane may include at least 25 wt %, or at least 40 wt % ZIF. In some cases, the membrane may include at least 80 wt % ZIF. The wt % of the ZIF is preferably determined as a % of the weight of the membrane.

The polymer of the matrix phase may include any appropriate material. For example, the polymer may comprise a silicone elastomer. For example, the matrix may comprise polydimethylsiloxane (PDMS), PMPS, PEBA, polyimide, polyamide and/or polysulphone.

The membrane may include a ZIF having a pore size of between from about 1.0 Å to about 10.0 Å. Preferably the pore size is determined as the diameter of the largest sphere which would fit into the pore.

The ZIF material may comprise particles, and the average particle size of the ZIF particles is at least 10 nm.

The average particle size of the ZIF particles may be for example at least 20 nm. Preferably, the ZIF material includes particles having a size in the range of from about 0.01 to 50 μm. Where the ZIF includes irregular or non-spherical particles, preferably the average size of the smallest dimension of the particles is at least 20 nm. Preferably the membrane includes ZIF particles having a smallest dimension at least 0.01 μm, and a largest dimension of not more than 50 μm. The particle size may be measured for example using SEM (Scanning Electron Microscopy) and/or TEM (Transmission Electron Microscopy) and/or DLS (Dynamic Light Scattering) and/or XRD (based on Scherrer's equation).

In some examples, the ZIF material includes particles having a size (at least in one direction in the particle) greater than the thickness of the matrix material. In other examples, the ZIF material includes some particles having a size greater, and some particles having a size smaller than the thickness of the matrix material of the membrane. In other examples, the ZIF material includes only particles having a size smaller than the thickness of the matrix material of the membrane.

The ZIF particles may include particles having a size (in at least one direction in the particle) greater than the thickness of the matrix material of the membrane.

The thickness of the membrane may be less than about 100 μm.

The thickness of the membrane may at least 1.0 μm, for example at least 1.5 μm, for example 2.0 μm or more. In examples, the thickness of the membrane may be from about 2 μm to 4 μm.

For some membranes, particularly for example those containing ZIF particles where the ZIF particles have a size less than the thickness of the matrix material, the thickness of the matrix material will be substantially equivalent to the thickness of the membrane. In other examples, the membrane itself may have a thickness greater than that of the matrix material.

A further aspect of the invention provides a process for separating an organic compound from an aqueous liquid mixture, the process comprising:

a) contacting the liquid mixture on one side of a mixed matrix pervaporation membrane to cause the organic compound to permeate the mixed matrix membrane, wherein the mixed matrix pervaporation membrane comprises

i) a matrix phase comprising a polymeric material, and

ii) a zeolitic imidazolate framework (ZIF) material dispersed in the matrix phase

wherein the thickness of the matrix phase of the membrane is greater than 0.5 μm and
b) removing from the other side of the membrane a permeate composition comprising a portion of the organic compound which permeated the membrane.

The process may include applying a vacuum for drawing the organic compound through the membrane.

The process may include providing a force to effect passage of the organic compound, for example an alcohol, through the membrane. For example a vacuum or negative pressure could be applied to draw the organic compound through the membrane. Alternatively or in addition, a sweep gas could be used. Other methods could be used as an alternative or in combination.

Aspects of the invention relate to the use of a membrane for the separation from a liquid mixture of any compound which includes carbon-containing molecules. Aspects of the invention find particular, but not exclusive, application in relation to organic compounds derived from a biological source, for example a plant source. Organic compounds which may be separated using membranes described herein include, but are not restricted to alcohols, aldehydes, esters, aromatic compounds and their derivatives.

Aspects of the invention find particular application for the separation of organic materials from an aqueous liquid mixture. The liquid mixture may additionally include other solvents and/or other components. The liquid mixture may include two or more organic compounds. Where the liquid mixture includes a plurality of organic compounds, the membranes of examples of aspects of the invention may be used for separating one or more of the organic compounds from the mixture.

For example, aspects of the invention may find application in the separation of one or more alcohols from an aqueous liquid mixture. Thus the organic compound may include an alcohol. For example, the alcohol may include butanol, for example iso butanol.

In some examples of the invention, the liquid mixture may comprise a fermentation broth, or be derived from a fermentation broth. Aspects of the invention find application in relation to the separation of an alcohol from a fermentation broth, for example in a fermentation process for the production of an alcohol.

A further aspect of the invention provides a mixed matrix pervaporation membrane comprising a zeolitic imidazolate framework (ZIF) material dispersed in a matrix phase comprising a polymer, wherein the thickness of the membrane is greater than 0.5 μm.

As discussed herein, the thickness of the membrane is of importance. It has been identified that relatively thin membranes, for example having a thickness of less than 0.5 μm, are more likely to exhibit undesirable swelling in use, and may be more stable than relatively thicker membranes.

Preferably the thickness of the membrane greater than 0.5 μm, preferably greater than 1.0 μm, for example greater than 2.0 μm.

The average ZIF particle size may be greater or smaller than the thickness of the matrix phase.

Preferably the thickness of the matrix phase is greater than 0.5 μM.

The ZIF may include one or more selected from the group comprising ZIF-4, ZIF-7, ZIF-8, ZIF-10, ZIF-22, ZIF-69, ZIF-78 and ZIF-90.

The membrane may include at least 1 wt % ZIF.

The membrane may include a ZIF having a pore size of between from about 1.0 Å to about 10.0 Å.

The average particle size of the ZIF particles may be at least 10 nm.

The thickness of the membrane may be less than about 100 μm.

An aspect of the invention provides a method of producing an alcohol from a fermentable hydrocarbon-containing composition, the method including the steps of:

a) fermenting the hydrocarbon-containing composition in the presence of a microbiological compound to form an alcohol-containing liquid mixture;
b) contacting the alcohol-containing mixture on one side of a mixed matrix pervaporation membrane for example as described herein and
c) removing from the other side of the membrane a permeate composition comprising a portion of the alcohol which permeated the membrane.

The method may produce more than one alcohol in the fermenting step, with one or more alcohols being removed in the permeate composition.

A further aspect of the invention provides a pervaporation apparatus for use in a system for separation of alcohol from a liquid mixture, the apparatus including a mixed matrix pervaporation membrane as described herein.

According to the invention there is further provided a method of preparing a pervaporation membrane for use in the separation of alcohol from a liquid mixture, the membrane including zeolitic imidazolate framework (ZIF) material dispersed in a matrix phase comprising a polymer, the method including the step of applying a solution to a substrate by dip coating the substrate into the solution, wherein the solution comprises the ZIF material and/or a precursor of the polymer.

Preferably the method of preparation of the membrane includes applying one or more compositions to a substrate. Preferably a dip coating method is used. After application of the material to the substrate, a heating or calcination step may be carried out. Dip coating steps may be carried out several times to form the required thickness of coating.

The solution may for example include ZIF particles. For example, ZIF particles may be applied to one or more surfaces of the substrate. For example the solution may comprise a mixture of ZIF particles in a liquid.

The solution may include a matrix material. For example, the matrix material may include a precursor of the polymer of the membrane matrix.

The solution may include ZIF particles and a precursor of the polymer. Thus both materials may be applied together to a substrate. For example, the ZIF particles may be blended with the polymer precursor to form a blend into which the substrate is dip coated. In alternative examples, the ZIF particles are first coated onto the substrate, followed by the application, for example by dip coating or other method, of the matrix material. In some examples, an array of ZIF particles is first applied to the substrate (for example by dip coating) followed by the application of matrix material. In some examples, the matrix material will at least partly fill gaps or interstices between the ZIF particles on the substrate surface.

Thus a further aspect of the invention provides, a method of preparing a pervaporation membrane for use in the separation of alcohol from a liquid mixture, the membrane including zeolitic imidazolate framework (ZIF) material dispersed in a matrix phase comprising a polymer, the method including the step of applying particles of the ZIF material to a surface of a substrate, and subsequently applying a precursor of the polymer to the particles of the ZIF material.

For example, the particles of ZIF material may be applied to the substrate by an appropriate coating method, for example by a spraying, or dip coating method, or by a mechanical method. For example the ZIF particles may be mechanically transferred onto the substrate by contact between the substrate and a source of ZIF material.

The substrate may comprise any appropriate material. For example, the substrate may comprise alumina. For use as a pervaporation membrane, it will not generally necessary to remove the mixed matrix membrane from the substrate. The substrate in examples provides mechanical support for the membrane.

A further aspect of the invention provides a process for separating an organic compound from an aqueous liquid mixture, the process comprising:

a) contacting the liquid mixture on one side of a mixed matrix pervaporation membrane to cause the organic compound to permeate the mixed matrix membrane, wherein the mixed matrix pervaporation membrane comprises

i) a matrix phase comprising a polymeric material, and

ii) a zeolitic imidazolate framework (ZIF) material dispersed in the matrix phase and

b) removing from the other side of the membrane a permeate composition comprising a portion of the organic compound which permeated the membrane.

A further aspect of the invention provides a mixed matrix pervaporation membrane comprising a zeolitic imidazolate framework (ZIF) material dispersed in a matrix phase comprising a polymer.

The invention extends to methods and/or apparatus and/or compositions and/or membranes being substantially as herein described with reference to any of the accompanying drawings.

Any feature in one aspect of the invention may be applied to other aspects of the invention, in any appropriate combination. In particular, features of method aspects may be applied to apparatus, composition or membrane aspects, and vice versa.

Preferred features of the present invention will now be described, purely by way of example, with reference to the accompanying drawings, in which:

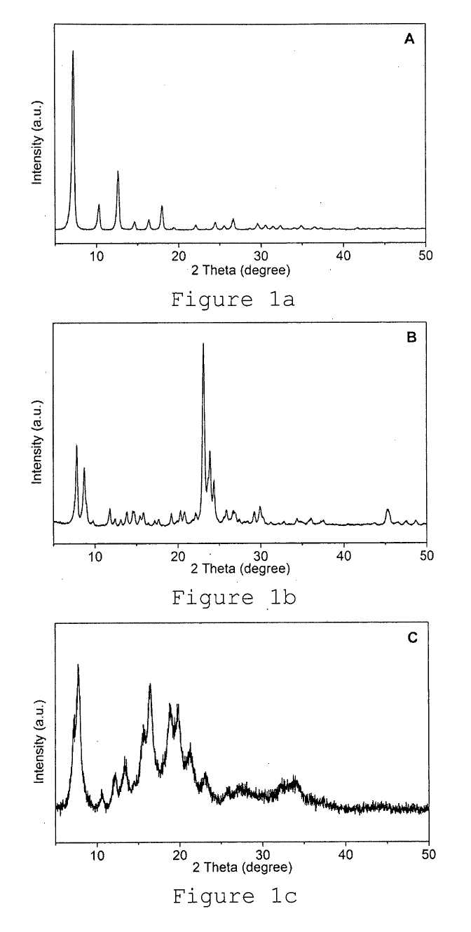

FIGS. 1a, 1b and 1c show examples of X-ray diffraction (XRD) patterns of ZIF-8 (FIG. 1a), silicalite-1 (FIG. 1b) and ZIF-7 (FIG. 1c) crystals;

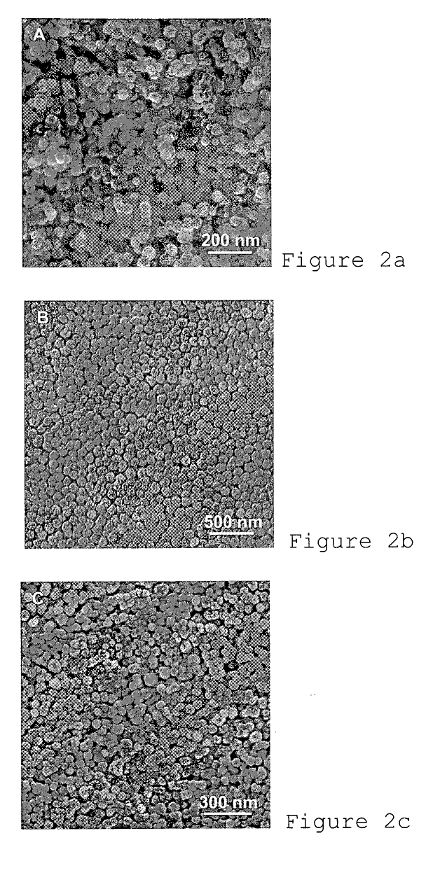

FIGS. 2a, 2b and 2c show SEM images of ZIF-8 (FIG. 2a), silicalite-1 (FIG. 2b) and ZIF-7 (FIG. 2c) crystals;

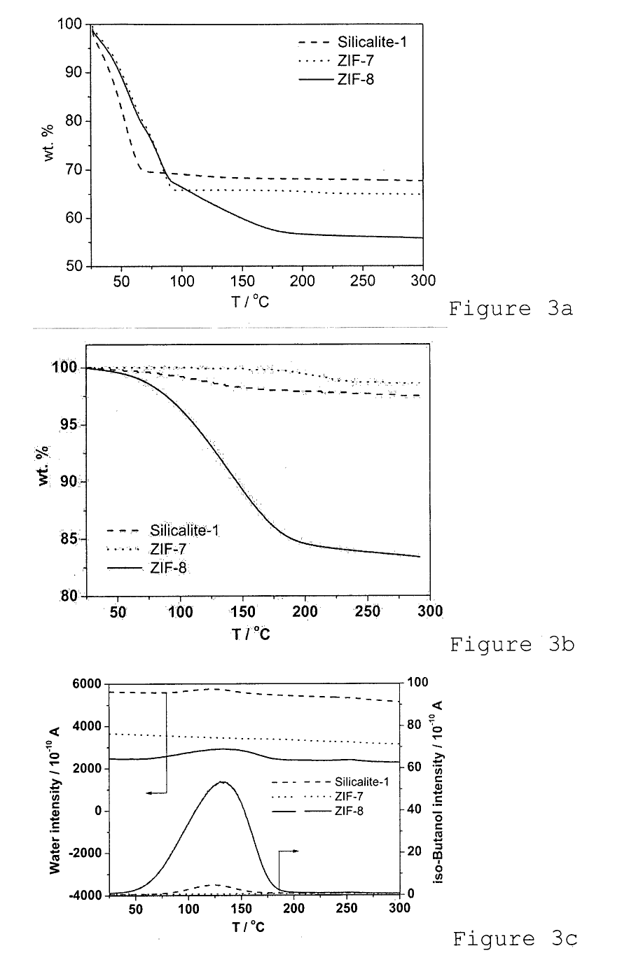

FIGS. 3a, 3b and 3c show thermogravity (TG) curves for desorption of iso-butanol and water from different adsorbents evaluated at room temperature for 1.5 h (FIG. 3a) and one week (FIG. 3b) coupled with MS curves (FIG. 3c);

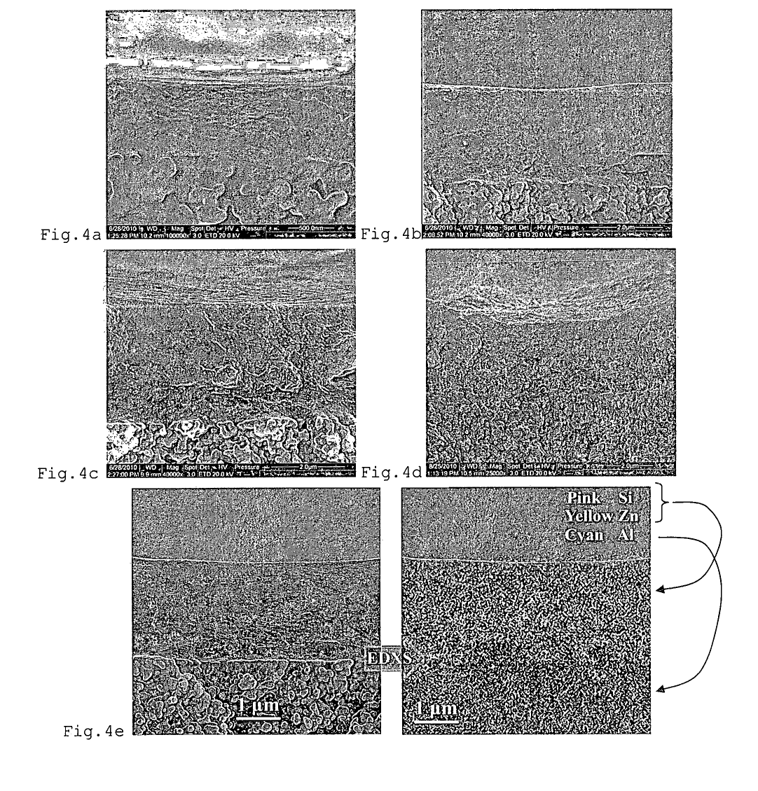

FIGS. 4a, 4b, 4c and 4d show SEM images of cross section of ZIF-8-PMPS-1 (M1) membranes with different W_{ZIF-8}/W_{PMPS} rate: (FIG. 4a) 5%; (FIG. 4b) 10%; (FIG. 4c) 15%; (FIG. 4d) 20%; and FIG. 4e shows the membrane cross section Energy-dispersive X-ray spectroscopy (EDXS) mapping;

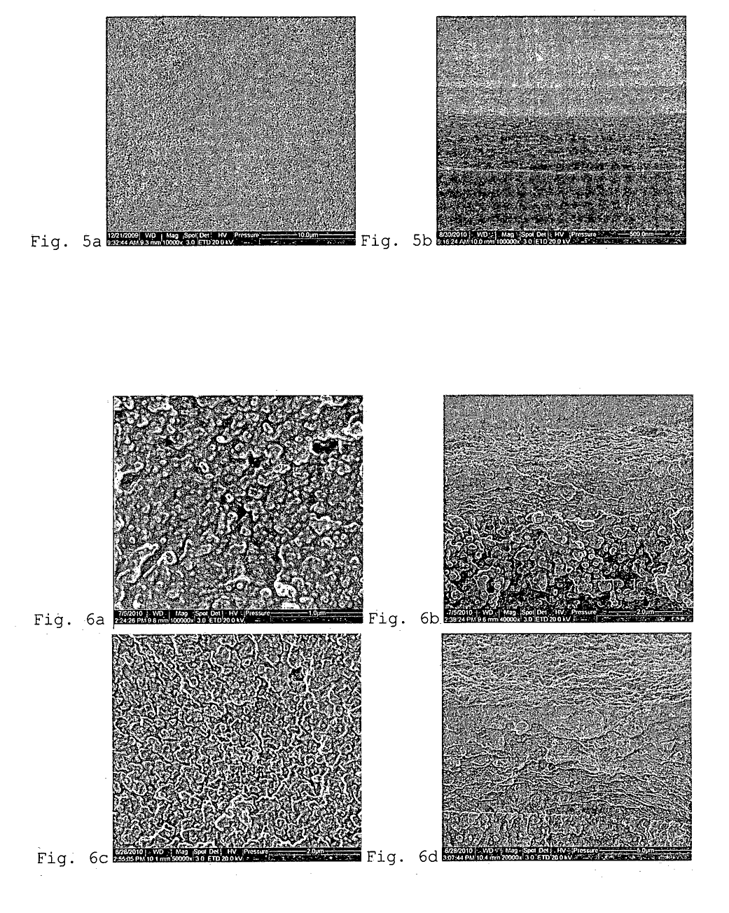

FIGS. 5a and 5b show SEM images of surface (FIG. 5a) and cross section (FIG. 5b) of PMPS membrane (M3);

FIGS. 6a, 6b, 6c and 6d show SEM images of ZIF-8-PDMS-1 (M4) membranes with different W_{ZIF-8} W_{PMPS} rate (weight ratio): (FIG. 6a) 5% surface; (FIG. 6b) 5% cross section; (FIG. 6c) 15% surface; (FIG. 6d) 15% cross section;

FIGS. 7a and 7b show SEM images of surface (FIG. 7a) and cross section (FIG. 7b) of ZIF-8-PMPS-2 membrane (M5);

FIGS. 8a and 8b show SEM images of surface (FIG. 8a) and cross section (FIG. 8b) of ZIF-8-PDMS-2 membrane (M6);

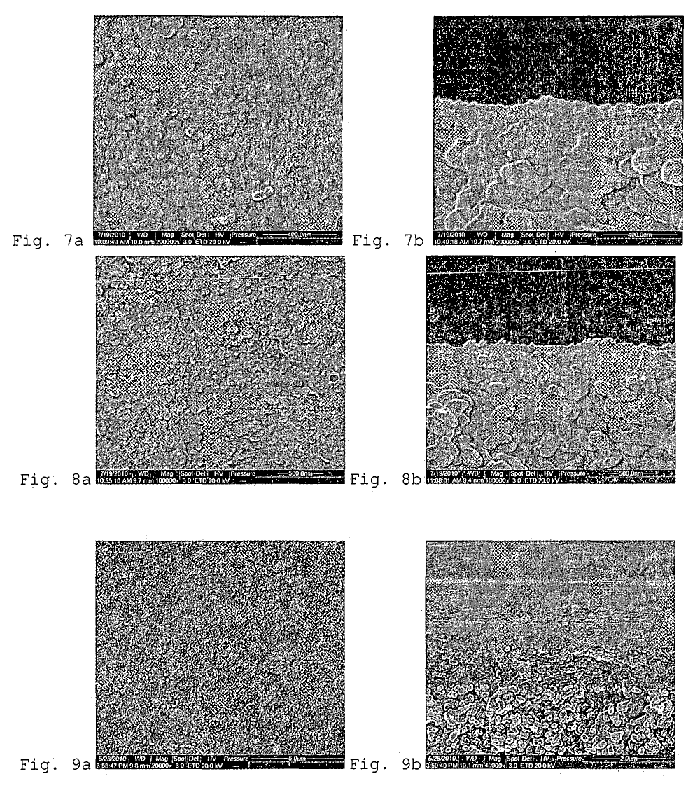

FIGS. 9a and 9b show SEM images of surface (FIG. 9a) and cross section (FIG. 9b) of ZIF-8-PEBA membrane (M7);

FIGS. 10a and 10b show SEM images of surface (FIG. 10a) and cross section (FIG. 10b) of PEBA membrane (M8);

FIGS. 10c and 10d show SEM images of a surface of a ZIF-8-PMPS-3 membrane (M9) at lower and higher magnification, respectively;

FIGS. 10e and 10f show SEM images of a surface of a ZIF-8-PMPS-4 membrane (M10) at lower and higher magnification, respectively;

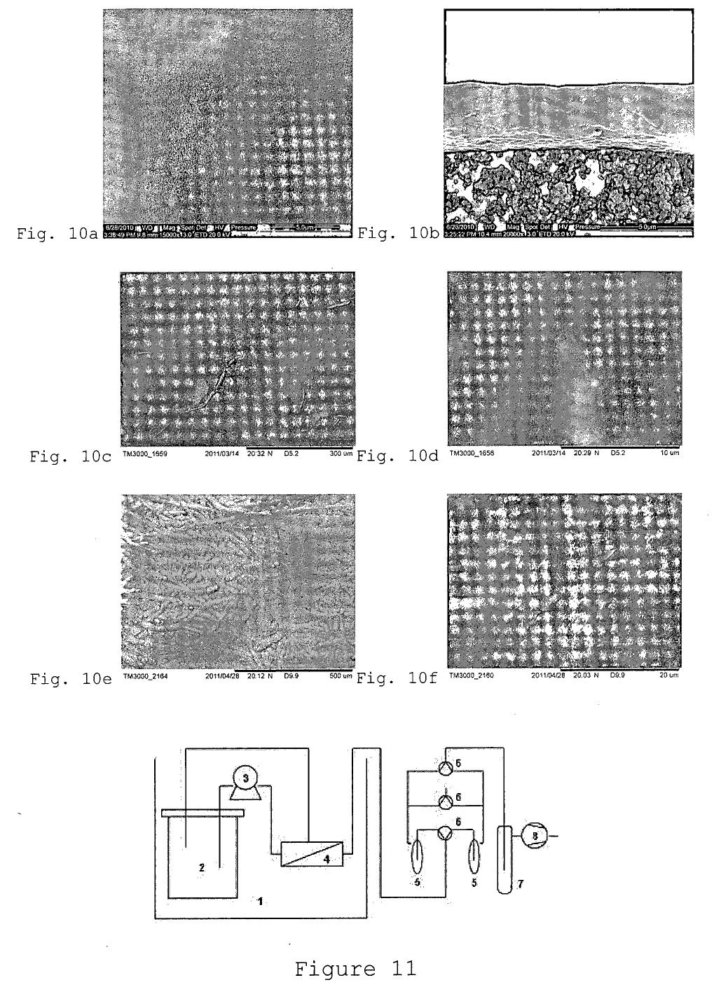

FIG. 11 shows a schematic diagram of an example of the pervaporation apparatus;

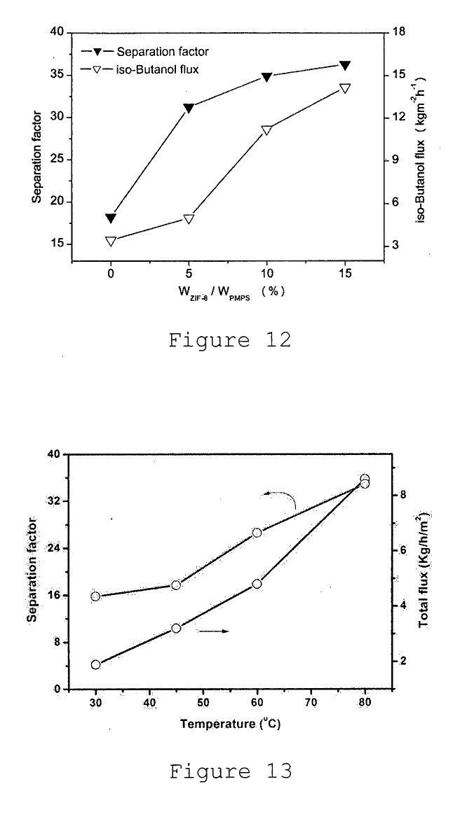

FIG. 12 is a graph showing the effect of ZIF-8 content on the performance (the fluxes were normalized to a membrane thickness of 1 μm) of the ZIF-8-PMPS-1 membranes (M1);

FIG. 13 is a graph showing the effect of temperature on the performance of membrane M1 with a feed composition of 3 wt. % i-butanol;

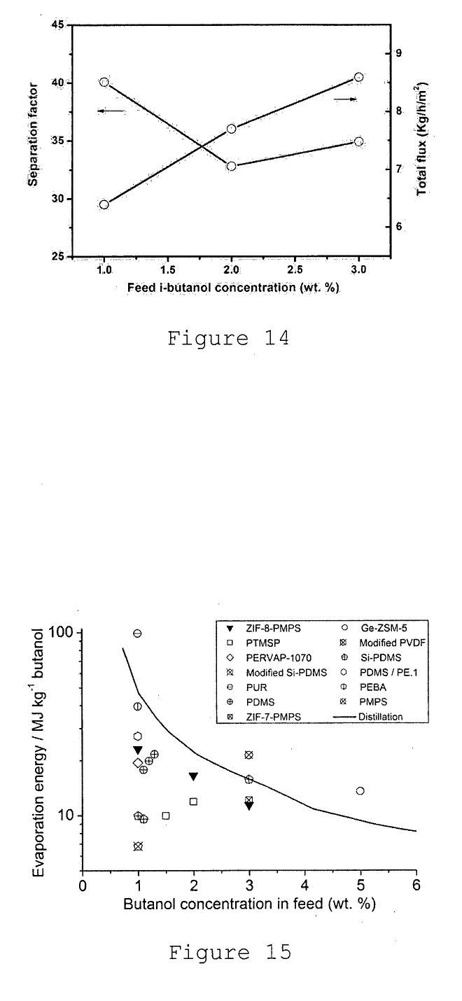

FIG. 14 is a graph showing the effect of concentration on the performance of membrane M1 at 80° C.;

FIG. 15 is a graph showing evaporation energy required to recover butanol from water as a function of the butanol feed concentration and butanol-water separation factor in an example. A distillation curve is shown for reference.

### EXAMPLE 1

### Preparation of Silicalite and ZIF Crystals

In this example, silicalite-1, ZIF-7, and ZIF-8 crystals were synthesized.

Silicalite-1 crystals were hydrothermally synthesized at relatively mild conditions using a modified recipe (Advanced Materials. 13 (2001) 1880-1883). The molar composition of synthetic solutions was kept at TPAOH:SiO₂:H₂O:EtOH being 9:25:408:100. First, 20 g of TPAOH (Aldrich, 20 wt. %) was mixed with 2.044 g H₂O under stirring. After formation of a homogenous solution, 11.386 g TEOS (Kermel, AR) was added and stirred at room temperature for 24 h. Subsequently, the clear solution was transferred to a Teflon-lined autoclave, heated to 90° C. and kept there for 24 h statically. After hydrothermal synthesis, the product was centrifuged at 15,000 rpm for 1 h and washed with water using ultrasonic for 1 h for several cycles.

ZIF-7 crystals were prepared by a modified recipe reported by Li et al. (Angewandte Chemim International Edition. 49, (2010) 548-551). 140 mL N,N-dimethylformamide (BoDi, AR) was added into a solid mixture of 0.423 g Zn(NO₃)₂.6H₂O and 1.077 g benzimidazole (Aldrich, 98%) with stirring. After being kept at room temperature for 23 h, the product was centrifugally separated and washed with methanol.

ZIF-8 crystals were synthesized according to the route reported by Cravillon et al. (Chemistry Materials. 21 (2009) 1410-1412). A solution of Zn(NO₃)₂.6H₂O (1.026 g, Aldrich, ≧99.0%) in 70 mL of methanol (BoDi, AR) was rapidly poured into a solution of 2-methylimidazole (2.072 g, Aldrich, 99%) in 70 mL of methanol under stirring. After 1 h, the crystals were separated from the mother liquid by centrifugation and washed with methanol.

### EXAMPLE 2

### Synthesis of Composite Membrane M1

In this example, a ZIF-8-PMPS-1 composite membrane (M1) was synthesized by a solution blending-dip-coating method.

Alumina capillary tubes (3.7 mm outside diameter, 2.4 mm inside diameter, 6 cm length, Hyflux Ltd.) were used as supports in the current example. The pore size of the inner surface was 40 nm. Before use, the tubes were sonicated for 5 min to remove impurities physically adsorbed on the surface, and then dried in an oven at 50° C. The outer surfaces of the supports were wrapped with Teflon® tapes.

To obtain composite membranes, ZIF-8 crystals were redispersed in iso-octane (Kermel, AR) using a probe-type sonicator (AiDaPu, Hangzhou Success Ultrasonic Equipment Co., Ltd.) with the horn immersed in the sample for 10 min in an ice bath. This solution (4.5 wt. %) was then allowed to heat to room temperature. Catalyst (dibutyltin dilaurate, Shanghai Resin Factory Co., Ltd.), curing agent (tetraethylorthosilicate, TEOS, Kermel, AR), iso-octane, α,ω-dihydroxypolymethylphenylsiloxane (PMPS, Shanghai Resin Factory Co., Ltd.) and the above ZIF-8 iso-octane solution were added in a glass bottle one after another (for the standard membrane, in the weight composition: Catalyst/TEOS/PMPS/ZIF-8/iso-octane=1:10:100:10:333). This mixture was sonicated for 5 min using the probe-type sonicator in an ice bath. The resulting mixture was homogeneous, and then kept at room temperature for 10 min. After that, the capillary tube was dip-coated into this mixture for 10 s and withdrawn at a speed of 1 mm/s using a dip-coater (WPTL0.01, Shenyang Kejing Auto-instrument Co., Ltd.). The membrane was cured at 25° C. for 24 h, 100° C. for 12 h and then kept at 100° C. for another 12 h under vacuum. The resulting membrane was ZIF-8-PMPS-1 composite membrane (M1).

### EXAMPLE 3

### Synthesis of Composite Membrane M2

In this example, ZIF-7-PMPS composite membrane (M2) was synthesized by solution blending-dip-coating method.

By substituting ZIF-8 with ZIF-7 crystals, the ZIF-7-PMPS membrane was prepared with same method as for M1 in Example 2.

### EXAMPLE 4

### Synthesis of Composite Membrane M3

In this comparative example, PMPS composite membrane (M3) was synthesized by dip-coating method.

### Preparation of γ-Al₂O₃ Sublayer.

A boehmite sol was prepared by peptization of boehmite suspension with 1.6 mol/L HNO₃ at 80° C. under stirring, and aged for 6 h. PVA 72000 and PEG 400 were used as additives of casting sol. The casting sol contained 2 wt. % PVA 72000, 1% PEG 400 and 0.5 mol/L boehmite. During dip-coating the sol, the ceramic support was contacted with sol, staying there for 9 s. After drying for 2 days at room temperature, the γ-Al₂O₃ layer was further calcined with a ramp speed of 0.5° C./min and held at 600° C. for 2 h (Science in China B. 40 (1997) 31-36).

### Preparation of Composite Membrane.

1 g of PMPS, 0.10 g of its curing agent and 0.050 g of catalyst were dissolved in 10.0 g of iso-octane by ultrasonic wave for 20 min. Then, the γ-Al₂O₃ modified tube was dip-coated into this solution for 10 s and withdrawn at a speed of 1.5 cm/s. After being dried at 20° C. for 10 min, the dip-coating process was repeated. Afterwards, the membrane was cured at 25° C. for 24 h, 50° C. for 3 h and then held at 50° C. for another 19 h under vacuum.

### EXAMPLE 5

### Synthesis of Composite Membrane M4

In this example, ZIF-8-PDMS-1 composite membrane (M4) was synthesized by solution blending-dip-coating method.

To obtain composite membranes, ZIF-8 crystals were redispersed in iso-octane using a probe-type sonicator with the horn immersed in the sample for 10 min in an ice bath. This solution (4.5 wt. %) was then allowed to heat to room temperature. iso-octane, PDMS (vinyl terminated) and its curing agent (methylhydrogen siloxane) (sylgard 184, used as received from Dow Corning Co.) and the above ZIF-8 iso-octane solution were added in a glass bottle one after another (for the standard membrane, the weight composition: curing agent/PDMS/ZIF-8/iso-octane=6:30:1.5:100). This mixture was sonicated for 5 min using the probe-type sonicator in an ice bath. The resulting mixture was homogeneous, and then kept at room temperature for 10 min. After that, the capillary tube was dip-coated into this mixture for 10 s and withdrawn at a speed of 1 mm/s using a dip-coater. The membrane was cured at 25° C. for 24 h, 100° C. for 12 h and then kept at 100° C. for another 12 h under vacuum.

### EXAMPLE 6

### Synthesis of Composite Membrane M5

In this example, ZIF-8-PMPS-2 composite membrane (M5) was synthesized by packing-filling method.

### Preparation of ZIF-8 Sublayer.

0.65 g of polyethyleneimine (PEI) (50 wt % in water, Aldrich) and 0.325 g of ZIF-8 was dissolved in 13 g of water using ultrasonic wave. After that, the capillary tube was dip-coated into this mixture for 20 s and withdrawn at a speed of 1 mm/s using a dip-coater. After briefly drying at room temperature for 2 h, the modified support was dried in an oven (80° C.) overnight.

### Preparation of Composite Membrane.

1.2 g of PMPS, 0.12 g of its curing agent and 0.012 g of catalyst were dissolved in 10.8 g of iso-octane by ultrasonic wave for 20 min. Then, the modified tube was dip-coated into this solution for 10 s and withdrawn at a speed of 1 mm/s. After being dried at 20° C. for 10 min, the dip-coating process was repeated. Afterwards, the membrane was cured at 25° C. for 12 h, 100° C. for 12 h and then held at 100° C. for another 12 h under vacuum.

### EXAMPLE 7

### Synthesis of Composite Membrane M6

In this example, ZIF-8-PDMS-2 composite membrane (M6) was synthesized by packing-filling method.

### Preparation of ZIF-8 Sublayer.

The preparation process was the same as the method of M5 in Example 6.

### Preparation of Composite Membrane.

1.2 g of PMPS, and 0.24 g of its curing agent were dissolved in 10.8 g of iso-octane by ultrasonic wave for 20 min. Then, the modified tube was dip-coated into this solution for 10 s and withdrawn at a speed of 1 mm/s. After being dried at 20° C. for 10 min, the dip-coating process was repeated. Afterwards, the membrane was cured at 25° C. for 12 h, 100° C. for 12 h and then held at 100° C. for another 12 h under vacuum.

### EXAMPLE 8

### Synthesis of Composite Membrane M7

In this example, ZIF-8-PEBA composite membrane (M7) was synthesized by solution blending-dip-coating method.

To obtain composite membranes, ZIF-8 crystals were redispersed in n-butanol using a probe-type sonicator with the horn immersed in the sample for 10 min. This solution (1.0 wt. %) was then allowed to cool to room temperature. n-Butanol, PEBA (poly(ether block amide), PEBAX2533, Arkema, France) and the above ZIF-8 n-butanol solution were added in a glass bottle one after another (for the standard membrane, the weight composition: PEBA/ZIF-8/n-butanol=5:1:100) This mixture was stirred for 24 h at 85° C. The resulting mixture was homogeneous, and then kept at room temperature for 24 h. After that, the capillary tube was dip-coated into this mixture for 10 s and withdrawn at a speed of 1 mm/s using a dip-coater. After being dried at 25° C. for 12 h, the dip-coating process was repeated. The membrane was dried at 25° C. for 12 h, 70° C. for 24 h and then kept at 50° C. for another 48 h under vacuum.

### EXAMPLE 9

### Synthesis of Composite Membrane M8

In this comparative example, PEBA composite membrane (M8) was synthesized by dip-coating method.

n-Butanol and PEBA were added in a glass bottle (for the standard membrane, the weight composition: PEBA/n-butanol=5:95) This mixture was stirred for 24 h at 85° C. The resulting mixture was homogeneous, and then kept at room temperature for 24 h. After that, the capillary tube was dip-coated into this mixture for 10 s and withdrawn at a speed of 1 mm/s using a dip-coater. After being dried at 25° C. for 12 h, the dip-coating process was repeated. The membrane was dried at 25° C. for 12 h, 70° C. for 24 h and then kept at 50° C. for another 48 h under vacuum.

### EXAMPLE 10

### Synthesis of Composite Membrane M9

In this example, ZIF-8-PMPS-3 composite membrane (M9) was synthesized by packing-filling method.

### Preparation of ZIF-8 Sublayer.

The ZIF-8 particles were dried under vacuum at 150° C. for 20 h and ground. After that the ZIF-8 particles were rubbed on the metallic net disc (BEKAERT 3AL³) by hand.

### Preparation of Composite Membrane.

1.0 g of PMPS, 0.10 g of its curing agent and 0.01 g of catalyst were dissolved in 4.0 g of iso-octane by ultrasonic wave for 5 min. Then, the above mentioned ZIF-8 rubbed disc was dip-coated into this solution for 10 s and withdrawn at a speed of 1 mm/s. After being dried at 25° C. for 10 min, the dip-coating process was repeated. Afterwards, the membrane was cured at 25° C. for 24 h, 100° C. for 12 h and then held at 100° C. for another 12 h under vacuum.

### EXAMPLE 11

### Synthesis of Composite Membrane M10

In this example, ZIF-8-PMPS-4 composite membrane (M10) was synthesized.

### Preparation of ZIF-8 Layer.

The ZIF-8 particles were dried under vacuum at 150° C. for 20 h and ground. Subsequently, the ZIF-8 particles were rubbed on the metallic net disc (BEKAERT 3AL³) by hand. A solid mixture of 1.078 g zinc chloride, 0.972 g of 2-methylimidazole and 0.54 g of sodium formate was dissolved in 80 ml methanol by ultrasonic treatment. The seeded disc was immersed into this solution and heated in a microwave oven at 100° C. for 4 h.

### Preparation of Composite Membrane.

1.0 g of PMPS, 0.10 g of its curing agent and 0.01 g of catalyst were dissolved in 4.0 g of iso-octane by ultrasonic wave for 5 min. Then, the above mentioned disc with ZIF-8 layer was dip-coated into this solution for 10 s and withdrawn at a speed of 1 mm/s. Afterwards, the membrane was cured at 25° C. for 24 h, 100° C. for 12 h and then held at 100° C. for another 12 h under vacuum.

### EXAMPLE 12

### Synthesis of Composite Membrane M11

In this example, ZIF-8-PMPS-5 composite membrane (M11) was synthesized.

### Preparation of ZIF-8 Layer.

A solid mixture of 1.078 g zinc chloride, 0.972 g of 2-methylimidazole and 0.54 g of sodium formate was dissolved in 80 ml methanol by ultrasonic treatment. An asymmetric titania disc (rutil/anatas compound, Inocermic, Germany) was immersed into this solution and heated in a microwave oven at 100° C. for 4 h.

### Preparation of Composite Membrane.

1.0 g of PMPS, 0.10 g of its curing agent and 0.01 g of catalyst were dissolved in 4.0 g of iso-octane by ultrasonic wave for 5 min. Then, the above mentioned disc with ZIF-8 layer was dip-coated into this solution for 10 s and withdrawn at a speed of 1 mm/s. Afterwards, the membrane was cured at 25° C. for 24 h, 100° C. for 12 h and then held at 100° C. for another 12 h under vacuum.

### EXAMPLE 13

### Characterisation of ZIF-8, Silicalite-1 and ZIF-7

In this example, the characterization of the ZIF-8, silicalite-1 and ZIF-7, crystals was studied.

### X-ray Diffraction

X-ray diffraction (XRD) patterns were recorded on Rigaku D/MAX 2500/PC apparatus (using Cu Kα radiation, λ=0.154 nm at 40 kV and 200 mA). The XRD patterns of ZIF-8 (SOD zeolite-type structure, 3.4 Å apertures size), silicalite-1 (MFI zeolite-type structure, 5.5 Å apertures size) and ZIF-7 (SOD zeolite-type structure, 3.0 Å apertures size) crystals are shown in FIGS. 1a to c, respectively, which indicate that the three types of crystals were successfully synthesized.

### Scanning Electron Microscopy

The as-made crystals were sputter coated with gold and their morphologies were studied by scanning electron microscopy (SEM, 200 FEG, FEI Co., 20 kV). Ultrafine ZIF-8 (FIG. 2a, 40±20 nm), silicalite-1 (FIG. 2b, 80±30 nm) and ZIF-7 (FIG. 2c, 80±30 nm) crystals were synthesized each with a narrow size distribution as seen in FIGS. 2a to c.

### Adsorption

Before the evaluation of adsorption, the washed ZIF-8 and ZIF-7 crystals were dried at 85° C. for 48 h under vacuum. The silicalite-1 was calcined at 600° C. for 10 h and then ground. 1.0 g of the ZIF-8, ZIF-7 and silicalite-1 nanocrystals were each separately dispersed in an alcohol-containing aqueous solution. Each alcohol was investigated separately, the nanocrystals being dispersed in 12.0 g of 3.0 wt. % alcohol (aqueous solution for ethanol, n-propanol and n-butanol as the alcohol, for n-pentanol the nanocrystals were dispersed in 24.0 g of 3.0 wt. % n-pentanol aqueous solution, using ultrasonic wave for 1 h, and then kept at room temperature for 1 day. 1.0 g of ZIF-8 was dispersed in 15.0 g of 5.0 wt % furfural alcohol aqueous solution employing the same method. The suspensions were centrifuged. The clear supernatants were removed and their compositions were determined by gas chromatography (GC, Agilent 7890). According to a depletion method, the amount of alcohol adsorbed was calculated from the difference in composition before and after contact with the adsorbent, assuming that only alcohol had been adsorbed. The ZIF-8 crystals were seen to selectively adsorb iso-butanol (kinetic diameter, 5.0 Å) from aqueous solution. The amount of iso-butanol adsorbed is 0.28 g/g ZIF-8 (Table 1), 5.6 times of that of the popular, organophilic silicalite-1 crystals which were widely used as fillers of alcohol (including butanol) recovery pervaporative composite membranes. Without wishing to be bound by any particular theory, it is thought that this adsorption performance of the ZIF-8 sample is due to factors including its strong hydrophobic character, very large surface area, flexibility induced increase of the aperture size for accepting large adsorbates. Although ZIF-7 and ZIF-8 have the same structure, ZIF-7 possesses narrower apertures size and more rigid framework compared with ZIF-8. So, as shown in Table 1, ZIF-7 exhibits almost no adsorption of iso-butanol.

TABLE 1
Characterization of different adsorbents
for iso-butanol adsorption from water.

| Adsorbent | Silicalite-1 | ZIF-7 | ZIF-8 |
|---|---|---|---|
| Amount adsorbed | 0.05 | 0.03 | 0.28 |
| (g/g adsorbent) | | | |

As shown in Table 2, it will be clear that all the tested alcohol can be selectively adsorbed. The amount adsorbed increases with the alcohol comprising more carbon atoms.

TABLE 2
Characterization of ZIF-8 for different
alcohol adsorption from water.

| | | n- | n- | n- | |
|---|---|---|---|---|---|
| Alcohol | Ethanol | Propanol | Butanol | Pentanol | Furfural |
| Amount adsorbed | 0.05 | 0.24 | 0.28 | 0.45 | 0.53 |
| (g/g adsorbent) | | | | | |

Thermal Gravimetric Coupled with Mass Spectrograph

After the evaluation of adsorption, the subnatant solids were wiped quickly with a filter paper, evaporated at room temperature for 1.5 h (FIG. 3a) and one week (FIG. 3b, c) respectively, then run on a Pyris Diamond TG/DTA thermal gravimetric analyzer coupled with MS (mass spectrograph, iso-butanol, m/z=43; water, m/z=18.) in a continuous flow of air with a temperature ramp of 10° C./min. FIG. 3a shows that the ZIF-8 exhibits two points of inflection in desorption step. As shown in FIG. 3b, after the further evaporation of the liquid in the bulk of the subnatant solids, only one inflection was observed in the desorption step. This indicates that the later inflection of ZIF-8 desorption curve in FIG. 3A related to the desorption of absorbates in the crystals channels. The MS curves indicate the obvious iso-butanol desorption from ZIF-8, silicalite-1 crystals channels, and almost no desorption from ZIF-7, which were consistent with the adsorption characterization (Table 1). Moreover, for ZIF-8, FIGS. 3a and b show an almost complete desorption of absorbates at 175° C. Without wishing to be bound by any particular theory, this suggests adsorption on ZIF-8 can be described as physical adsorption. These properties are believed make ZIF-8 a good substitute for silicalite-1 for using as a filler in polymer composite membranes for pervaporative recovery of alcohols, for example of butanol from aqueous solution.

### EXAMPLE 14

### Characterization of Membranes (M1-M11)

### Scanning Electron Microscopy

The as-made membranes were sputter coated with gold and their morphologies were studied by scanning electron microscopy (SEM, 200 FEG, FEI Co., 20 kV).

FIGS. 4a, 4b, 4c and 4d show SEM images of cross section of ZIF-8-PMPS-1 (M1) membranes with different W_{ZIF-8}/W_{PMPS} rate: (FIG. 4a) 5%; (FIG. 4b) 10%; (FIG. 4c) 15%; (FIG. 4d) 20%. As shown in FIG. 4 (Membrane M1), the ZIF-8 crystals are embedded in the PMPS phase with few or no interfacial voids. The membranes are about 1.0-4.0 μm thick offering the possibility to achieve a very high flux for pervaporation. As shown in the membrane cross section Energy-dispersive X-ray spectroscopy (EDXS) mapping (FIG. 4e), there is a sharp transition between the ZIF-8 nanocrystals (Zn signal) and the alumina support (Al signal). The intrusion of PMPS (Si signal) into the support can be observed, which is advantageous in terms of increasing the structure stability.

FIGS. 5a and 5b show SEM images of a surface (FIG. 5a) and cross section (FIG. 5b) of PMPS membrane (M3). As shown in FIG. 5 (M3), homogeneous thin (about 1 μm thick) PMPS membrane was fabricated on the γ-Al₂O₃ layer. This membrane was prepared for performance comparison with that of M1.

FIGS. 6a, 6b, 6c and 6d show SEM images of ZIF-8-PDMS-1 (M4) membranes with different W_{ZIF-8}/W_{PMPS} rate: (FIG. 6a) 5% surface; (FIG. 6b) 5% cross section; (FIG. 6c) 15% surface; (FIG. 6d) 15% cross section. As shown in FIG. 6 (M4), ZIF-8-PDMS membranes were attached on the support. It can be observed that ZIF-8 crystals were embedded in the PDMS phase. The membranes are about 2.0-6.0 μm thick.

FIGS. 7a and 7b show SEM images of surface (FIG. 7a) and cross section (FIG. 7b) of ZIF-8-PMPS-2 membrane (M5). FIGS. 8a and 8b show SEM images of surface (FIG. 8a) and cross section (FIG. 8b) of ZIF-8-PDMS-2 membrane (M6). As shown in FIGS. 7 (M5) and 8 (M6), From the SEM top view, the texture of the preformed ZIF-8 layer can still be distinguishable, suggesting a very thin layer of polymer on the ZIF sub-layer in these examples. From cross-sectional views, it can be seen that the thickness of the composite membrane is almost the same as that of the dip-coated ZIF-8 layer (about 300 nm). The so-obtained nanocomposite membrane has a high particle loading of about 74 vol %, as calculated using closest packing model. The polymer filled the interspaces between the inorganic particles and covered the surface of them uniformly. No voids between the inorganic particles and polymer were observed, suggesting good particle-polymer contact.

FIGS. 9a and 9b show SEM images of surface (FIG. 9a) and cross section (FIG. 9b) of ZIF-8-PEBA membrane (M7). As shown in FIG. 9 (M7), the ZIF-8 crystals are embedded in the PEBA phase. The membrane is about 3 μm thick offering the possibility to achieve a very high flux for pervaporation.

FIGS. 10a and 10b show SEM images of surface (FIG. 10a) and cross section (FIG. 10b) of PEBA membrane (M8). As shown in FIG. 10 (M8), homogeneous thin (about 5 μm thick) PEBA membrane was fabricated on the support. This membrane was prepared for the performance comparison with that of M7.

FIGS. 10c and 10d show SEM images of surface of ZIF-8-PMPS-3 membrane (M9). The close-packed ZIF-8 particles were embedded in the interspaces of the metallic net uniformly and linked with the polymer phase. No voids of the composite membrane were observed, suggesting good particle-polymer-net contact.

FIGS. 10e and 10f show SEM images of surface of ZIF-8-PMPS-3 membrane (M10). The large ZIF-8 particles (10 μm) grow on the metallic net and the interspaces among them were filled with polymer phase.

### EXAMPLE 15

### Measurement of Properties of M1 to M11

The properties of the composite membranes (M1-M8) were measured under different conditions.

The pervaporation apparatus utilized is shown schematically in FIG. 11. The pervaporation apparatus includes a feed tank 2 supplying feed via a pump 3 to a membrane module 4 including the pervaporative membrane prepared as described above. The retentate from the membrane module 4 is recycled to the feed tank 2. The permeate from the membrane module 4 is passed to a three-way valve 6 from where it is fed to one of two cold traps 5. Downstream of the traps is arranged further three-way valves 6 and a buffer vessel 7. The permeate is drawn through the apparatus by a vacuum pump 8.

The properties of the as-synthesized tubular membranes were evaluated by pervaporative recovery of organics from aqueous solution. The effective membrane area was about 3.75 cm² and the permeation side was kept under vacuum. The permeation flux (J) was measured by weighing the condensed permeate:

J=W/(At)

where W refers to the weight of permeate (kg), A the membrane area (m²), t the time (h) for the sample collection. Permeate and feed concentrations were measured by off-line. The separation factor was determined as

α_{organic/water}=(Y_{organic}/(1−Y_{organic}))/(X_{organic}/(1−X_{organic}))

where X_{organic} and Y_{organic} denote the mass fraction of organic compounds in the feed and permeate sides, respectively. In most cases, for separating butanol, furfural or pentanol, the pervaporated condensate separated into two phases. In order to measure the concentration of organics in the condensate, the permeate was diluted with water to generate a single phase.
The pervaporation performance of composite membranes for recovering organics from its aqueous solution (1-3 wt. %, 353 K) is shown in Table 3. All the tested membranes can selectively separate iso-butanol from water. The ZIF-8-PMPS-1 membrane (M1) was seen to possess the highest separation factor with very high iso-butanol flux (4.5 kgm⁻²h⁻¹). The ZIF-8-PMPS-3 membrane (M9) shows good performance for recovering furfural.

It can be seen that the separation factor for the relatively thin membranes M5 and M6, both of which have a thickness less than 500 nm, was significantly less than for the other, thicker membranes.

TABLE 3
pervaporation performance of composite membranes for recovering
iso-butanol from its aqueous solution (3 wt. %, 353K).

| | Total Flux | iso-Butanol Flux | Separation |
|---|---|---|---|
| Membrane | (kgm⁻²h⁻¹) | (kgm⁻²h⁻¹) | Factor |
| ZIF-8-PMPS-1 (M1) | 8.6 | 4.5 | 34.9 |
| ZIF-7-PMPS (M2) | 6.1 | 3.1 | 32.7 |
| PMPS (M3) | 15.2 | 5.5 | 18.2 |
| ZIF-8-PDMS-1 (M4) | 12.9 | 5.7 | 25.2 |
| ZIF-8-PMPS-2 (M5) ^{a} | 20.0 | 4.5 | 9.3 |
| ZIF-8-PDMS-2 (M6) ^{a} | 19.3 | 4.6 | 10.0 |
| ZIF-8-PEBA (M7) | 17.1 | 6.6 | 20.4 |
| PEBA (M8) | 15.6 | 5.7 | 18.6 |
| ZIF-8-PMPS-3 (M9) | 1.0 | 0.38 | 19.9 |
| ZIF-8-PMPS-3 (M9) ^{b} | 0.64 | 0.15 | 31.5 |
| ZIF-8-PMPS-4 (M10) | 1.0 | 0.24 | 15.0 |
| ZIF-8-PMPS-5 (M11) | 0.8 | 0.30 | 19.0 |

^{a} The membranes were destroyed after being tested for half an hour.
^{b} Feed: 1 wt. % furfural aqueous solution, 353K.

Three ZIF-8-PMPS-1 membranes (M1) with different W_{ZIF-8}/W_{PMPS} values (weight ratio) were prepared. The dense pure PMPS membrane was difficult to construct on the alumina capillary directly, and thus it was synthesized on the support modified by γ-Al₂O₃ layer (M2). To determine the variability in performance due to intrinsic properties of membranes, the fluxes presented in FIG. 12 were normalized to a thickness of 1 assuming that an inverse proportionality between the flux and the membrane thickness. As the ZIF-8 concentration in PMPS increases, the separation factor and iso-butanol flux increase simultaneously and significantly (FIG. 12). This phenomenon corresponds with the desired result that the ZIF-8 creates a preferential pathway for iso-butanol permeation in virtue of adsorption selectivity, forcing water to primarily transport through the polymer phase.

For M1, FIG. 13 shows that at a feed composition of 3 wt. % iso-butanol, both flux and separation factor increased with temperature. Without wishing to be bound by any particular theory, this is thought to be due to the increase of mobility of permeating molecules, which is enhanced by the temperature and the higher mobility of the polymer segments, as well as the increase of desorption rate of iso-butanol in ZIF-8 particles. The activation energy of iso-butanol permeation is higher than that for water, so the separation factor increases with an increase of temperature. The results suggested that the composite membrane showed little swelling even at higher temperature. This may be due to the effects of cross-linking of ZIF-8 and polymer.

For M1, FIG. 14 shows the effect of iso-butanol concentration on separation factor and total flux. Without wishing to be bound by any particular theory, it is thought that by increasing the iso-butanol concentration, iso-butanol in the feed phase had more sorption interaction with the membrane phase due to the affinity of iso-butanol being higher than water to the membrane. Furthermore, the sorption of the iso-butanol may increase the free volume and chain mobility of the polymer. Consequently, the diffusion of water through the membrane can be enhanced. Therefore, that the flux increases significantly with an increase in feed iso-butanol concentration is understandable. The denominator term in the selectivity relationship becomes large at high feed iso-butanol concentrations, thus giving low separation factor.

As shown in Table 4, M1 and M3 can selectively separate ethanol, n-propanol, n-butanol and n-pentanol from water. Both separation factor and total flux increased with the alcohol comprising more carbon atom (excluding the separation factor of n-pentanol on M1). Compared with M3, M1 possesses higher separation factor and flux corresponding to the tested alcohol for most of the alcohols in the experiment. This phenomenon is consistent with the desired result that the ZIF-8 creates a preferential pathway for alcohol permeation in virtue of adsorption selectivity, forcing water to primarily transport through the polymer phase. Compared with the light alcohol, the little drop of separation factor and flux for separating n-pentanol might be due to its larger kinetic diameter which induced a lower transport rate.

TABLE 4
Pervaporation performance of the PMPS and ZIF-
8-PMPS membranes for different alcohol recovery
at 80° C. with feed concentration of 1 wt. %.

| | | | n- | n- | n- |
|---|---|---|---|---|---|
| | Alcohol | Ethanol | Propanol | Butanol | Pentanol |
| Separation | PMPS (M3) | 5.4 | 11.4 | 17.7 | 26.4 |
| factor | ZIF-8-PMPS-1 | 12.0 | 24.9 | 36.8 | 30.6 |
| | (M1) | | | | |
| Total flux | PMPS (M3) | 3.7 | 3.9 | 4.8 | 7.1 |
| (kgm⁻²h⁻¹) | ZIF-8-PMPS | 4.0 | 4.8 | 5.1 | 6.0 |
| | (M1) | | | | |

### EXAMPLE 16

In this example, an economic appraisal for recovering butanol from water was carried out. The energy required to evaporate permeate in a pervaporation process, normalized per unit of butanol permeated is calculated as follows (Journal of Chemical Technology and Biotechnology. 80 (2005) 603-629):

$${Q}_{norm}^{evap} = \frac{{\sum }_{i} {H}_{i}^{evap} {F}_{i}}{{F}_{BuOH}}$$

Where Hᵢ^{evap} is the heat of evaporation of species i, and Fᵢ is the flux of species i. When butanol and water dominate the feed and the permeate, the above equation can be rewritten in terms of feed butanol concentration (wt. %, C_{BuOH}^{feed}) and butanol-water separation factor (α) as:

$${Q}_{norm}^{evap} = {H}_{BuOH}^{evap} + {H}_{water}^{evap} ( \frac{1 - {C}_{BuOH}^{feed}}{α {C}_{BuOH}^{feed}} )$$

The residual is fixed at 0.02 wt. % butanol. The energy required to further purify the permeate is ignored for this example. (The energy required by distillation to purify a solution containing 40 wt. % butanol is about 4% of that of a solution containing 0.5 wt. % butanol.) The condensation heat recovery which reduces pervaporation energy usage by 67% is not considered in this example.
The standard membrane (M1) developed in this study possesses separation factor of 34.9-40.1 with 1-3 wt. % feed iso-butanol aqueous solution at 80° C., which is among those with high separation performance of the reported membranes (Table 5).

TABLE 5
Pervaporation performance comparison (with the literature
reported membranes) for butanol recovery.

| | Feed | | | |
|---|---|---|---|---|
| | concen- | Feed | Total | |
| | tration | temperature | flux | Separation |
| Membrane | (wt. %) | (° C.) | (kgm⁻²h⁻¹) | factor |
| Ge-ZSM-5 | 5^{a} | 30 | 0.02 | 19.0 |
| PTMSP | 2^{a} | 37 | 0.59 | 46.3 |
| | 1.5^{a} | 70 | 1.03 | 70 |
| Modified PVDF | 2.5^{a} | 40 | 0.99 | 7.4 |
| PERVAP-1070 | 1^{a} | 70 | 0.34 | 47.8 |
| | 1^{a} | 70 | 0.61 | 93.0 |
| Si-PDMS | 1^{a} | 70 | 0.11 | 96.0 |
| | 3^{b} | 80 | 11.2 | 25.0 |
| Modified Si-PDMS | 1^{a} | 50 | 0.22 | 145 |
| PDMS/PE.1 | 1^{a} | 37 | 0.10 | 34 |
| PUR | 1^{b} | 50 | 0.08 | 9.2 |
| PEBA | 1^{b} | 50 | 0.24 | 23.2 |
| | 1^{a} | 70 | 0.35 | 48.7 |
| PDMS | 1^{b} | 50 | 0.07 | 40.0 |
| | 1^{a} | 50 | 0.07 | 35.0 |
| PMPS (M3) | 3^{b} | 80 | 15.2 | 18.2 |
| ZIF-7-PMPS (M2) | 3^{b} | 80 | 6.1 | 32.7 |
| ZIF-8-PMPS-1 (M1) | 3^{b} | 80 | 8.6 | 34.9 |
| | 1^{b} | 80 | 6.4 | 40.1 |

^{a}n-Butanol aqueous solution.
^{b}iso-Butanol aqueous solution.

As shown in FIG. 15, the evaporation energy for the standard membrane is clearly less than that of distillation, indicating that the replacement of the energy intensive distillation with pervaporation process is available. The total flux of the standard membrane is 6.4-8.6 kgm⁻²h⁻¹, which is significantly higher than that of the reported membranes (Table 5). Without wishing to be bound by any particular theory, it is thought that this is probably due to the thin and very homogeneous active layer and very low support resistant of the capillary. The ultrahigh flux translates into low membrane area for recovering butanol per unit weight, which in turn leads to less capital investments. So, the membrane shows good potential application in pervaporation process.

Aspects of the invention relate to metal-organic frameworks (MOFs) based pervaporation membranes, methods for their manufacture and their applications. Aspects of the invention relate to organic-inorganic membranes, and in particular examples to membranes for use in pervaporation for recovering alcohol, for example butanol, from solutions.

It will be understood that the present invention has been described above purely by way of example, and modification of detail can be made within the scope of the invention. Each feature disclosed in the description, and (where appropriate) the claims and drawings may be provided independently or in any appropriate combination.

1-23. (canceled)

24. A process for separating an organic compound from an aqueous liquid mixture, the process comprising:
a) contacting the liquid mixture on one side of a mixed matrix pervaporation membrane to cause the organic compound to permeate the mixed matrix membrane, wherein the mixed matrix pervaporation membrane comprises
i) a matrix phase comprising a polymeric material, and
ii) a zeolitic imidazolate framework (ZIF) dispersed in the matrix phase
wherein the thickness of the mixed matrix pervaporation membrane is greater than 0.5 μm and
b) removing from the other side of the membrane a permeate composition comprising a portion of the organic compound which permeated the membrane.

25. A process according to claim 24, wherein the membrane includes one or more ZIFs selected from the hydrophobic group comprising ZIF-1, ZIF-2, ZIF-3, ZIF-4, ZIF-5, ZIF-6, ZIF-7, ZIF-8, ZIF-9, ZIF-10, ZIF-11, ZIF-12, ZIF-14, ZIF-20, ZIF-21, ZIF-22, ZIF-23, ZIF-25, ZIF-60, ZIF-61, ZIF-62, ZIF-63, ZIF-64, ZIF-65, ZIF-66, ZIF-67, ZIF-68, ZIF-69, ZIF-70, ZIF-71, ZIF-72, ZIF-73, ZIF-74, ZIF-75, ZIF-76 ZIF-78, ZIF-91, ZIF-92, ZIF-93, ZIF-96, ZIF-97 and ZIF-100.

26. A process according to claim 24, wherein the membrane includes at least 1 wt % ZIF.

27. A process according to claim 24, wherein the membrane includes a ZIF having a pore size of between from about 1.0 Å to about 10.0 Å.

28. A process according to claim 24, wherein the ZIF material comprises particles, and the average particle size of the ZIF particles is at least 10 nm.

29. A process according to claim 24, wherein the ZIF particles include particles having a size greater than the thickness of the matrix material of the membrane.

30. A process according to claim 24, wherein the thickness of the membrane is less than about 100 μm.

31. A process according to claim 24, including applying a vacuum for drawing the organic compound through the membrane.

32. A process according to claim 24, wherein the organic compound includes an alcohol.

33. A process according to claim 32, wherein the alcohol comprises butanol.

34. A mixed matrix pervaporation membrane comprising a zeolitic imidazolate framework (ZIF) material dispersed in a matrix phase comprising a polymer, wherein the thickness of the membrane is greater than 0.5 μm.

35. A membrane according to claim 34, wherein the ZIF includes one or more selected from the group comprising ZIF-4, ZIF-7, ZIF-8, ZIF-10, ZIF-22, ZIF-69, ZIF-78 and ZIF-90.

36. A membrane according to claim 34, wherein the membrane includes at least 1 wt % ZIF.

37. A membrane according to claim 34, wherein the membrane includes a ZIF having a pore size of between from about 1.0 Å to about 10.0 Å.

38. A membrane according to claim 24, wherein the average particle size of the ZIF particles is at least 10 nm.

39. A membrane according to claim 24, wherein the thickness of the membrane is less than about 100 μm.

40. A method of producing an alcohol from a fermentable hydrocarbon-containing composition, the method including the steps of:
a) fermenting the hydrocarbon-containing composition in the presence of a microbiological compound to form an alcohol-containing liquid mixture;
b) contacting the alcohol-containing mixture on one side of a mixed matrix pervaporation membrane according to claim 34 and
c) removing from the other side of the membrane a permeate composition comprising a portion of the at least one alcohol which permeated the membrane.

41. Pervaporation apparatus for use in a system for separation of alcohol from a liquid mixture, the apparatus including a mixed matrix pervaporation membrane according to claim 34.

42. A method of preparing a pervaporation membrane for use in the separation of alcohol from a liquid mixture, the membrane including zeolitic imidazolate framework (ZIF) material dispersed in a matrix phase comprising a polymer, the method including the step of applying a solution to a substrate by dip coating the substrate into the solution, wherein the solution comprises the ZIF material and/or a precursor of the polymer.

43. A method according to claim 42, wherein the solution includes ZIF particles.

44. A method according to claim 42, wherein the solution includes a matrix material.

45. A method according to claim 43, wherein the solution includes ZIF particles and a precursor of the polymer.

46. A method of preparing a pervaporation membrane for use in the separation of alcohol from a liquid mixture, the membrane including zeolitic imidazolate framework (ZIF) material dispersed in a matrix phase comprising a polymer, the method including the step of applying particles of the ZIF material to a surface of a substrate, and subsequently applying a precursor of the polymer to the particles of the ZIF material.
